(19) United States
(12) Patent Application Publication
(10) Pub. No.: US 2025/0275939 A1
(43) Pub. Date: Sep. 4, 2025

(54) CO-CRYSTALS

(71) Applicant: UNIVERSITY OF IOWA RESEARCH FOUNDATION, Iowa City, IA (US)

(72) Inventors: Sanika Jadhav, Iowa City, IA (US); Lewis Stevens, Iowa City, IA (US)

(73) Assignee: UNIVERSITY OF IOWA RESEARCH FOUNDATION, Iowa City, IA (US)

(21) Appl. No.: 19/067,148

(22) Filed: Feb. 28, 2025

Related U.S. Application Data
(60) Provisional application No. 63/560,162, filed on Mar. 1, 2024.

(51) Int. Cl. A61K 31/353; A61K 9/20; A61K 9/48; A61K 47/02; A61K 47/22

(52) U.S. Cl. CPC A61K 31/353; A61K 9/2018; A61K 9/2027; A61K 9/2054; A61K 9/2059; A61K 9/485; A61K 9/4858; A61K 9/4866; A61K 47/02; A61K 47/22

(57) ABSTRACT

The invention provides co-crystals comprising a flavonoid and a conformer. The co-crystals have improved properties (e.g. solubility, formulatability, and/or bioavailability) compared to the parent flavonoid. The co-crystals are useful for medical therapy.

### CROSS-REFERENCE TO RELATED APPLICATION

This application claims priority to U.S. Provisional Application No. 63/560,162 that was filed on 1 Mar. 2024. The entire content of the application referenced above is hereby incorporated by reference herein.

### BACKGROUND

Flavonoids are a class of secondary metabolites found in plants and commonly used in human diets. Research has shown that flavonoids have a range of health benefits, including antioxidant, anti-inflammatory and anti-cancer properties. They have been linked to a reduced risk of chronic diseases such as cardiovascular disease, as well as improved cognitive function in Alzheimer's disease. The best way to get flavonoids into your system is directly from food, mainly berries, citrus fruits, and drink sources such as tea and wine. Despite having such a great potential to improve many health conditions one of the biggest reasons for its poor therapeutic efficacy is poor water solubility. The interaction of molecules in a solid state and their interaction with water dictates the solution behavior of the compound. Flavonoids have complex molecular structures with typically more than 15-carbon skeleton and thus have minimal interaction with water.

Currently there is a need for more highly soluble forms of flavonoids. More highly-soluble forms of flavonoids would be useful as therapeutic agents.

### SUMMARY

Solid forms of flavonoids have been prepared through a ‘co-crystallization’ approach. These solid forms have different intermolecular interactions in solid state, improved interaction with water, and improved solubility. Co-crystals are molecular complex of crystalline drug/flavonoid with another compound in a single solid form. The end-product is in solid form and can be easily manufactured into tablets/capsules, unlike the majority of nanotechnology-based methods. Moreover, flavonoids have been combined with other bioactive molecules such as tetramethyl pyrazine (TMP) in the same solid form. Ligustrazine/TMP, a vital alkaloid in the traditional Chinese medicine Rhizoma Chuanxiong, which exerts therapeutic benefits such as antioxidation, anti-inflammatory effects, and neuroprotection. The combination of two bioactive molecules in one solid form is beneficial for treating diseases such as Alzheimer's disease.

New solid forms of naringenin, hesperetin, and quercetin have been generated. The coformers used in these co-crystals are more than 100-1000 times more water soluble than the flavonoids, thus there is a great potential to improve the solubility and in turn oral bioavailability of co-crystalized flavonoids by virtue of the high intrinsic solubility of coformers.

In one aspect the present invention provides a co-crystal comprising a flavonoid other than naringenin and a co-crystal former.

In another aspect the present invention provides a co-crystal comprising a flavonoid and a co-crystal former selected from the group consisting of succinimide, tetramethyl pyrazine, aspartame, hexamine, and betaine methanol.

The invention also provides a pharmaceutical composition comprising a co-crystal of the invention and a pharmaceutically acceptable excipient.

The invention also provides a method for treating or preventing a disease in an animal (e.g., a mammal such as a human) comprising administering a co-crystal of the invention to the animal.

The invention also provides a co-crystal of the invention for use in medical therapy. The invention also provides a method for treating cancer in an animal comprising administering a co-crystal of the invention to the animal, wherein the co-crystal has anticancer properties.

The invention also provides a method for producing an antioxidant effect in an animal in need of such treatment comprising administering a co-crystal of the invention to the animal, wherein the co-crystal has antioxidant properties.

The invention also provides a method for producing an anti-inflammatory effect in an animal in need of such treatment comprising administering a co-crystal of the invention to the animal, wherein the co-crystal has anti-inflammatory properties.

The invention also provides a method for treating cardiovascular disease in an animal comprising administering a co-crystal of the invention to the animal, wherein the co-crystal treats cardiovascular disease.

The invention also provides a method for improving cognitive function in an animal in need of such treatment comprising administering a co-crystal of the invention to the animal, wherein the co-crystal has cognitive enhancing properties

The invention also provides a method for treating Alzheimer's disease in an animal comprising administering a co-crystal of the invention to the animal, wherein the co-crystal treats Alzheimer's disease.

The invention also provides a co-crystal of the invention for the prophylactic or therapeutic treatment of cancer, wherein the co-crystal has anticancer properties.

The invention also provides a co-crystal of the invention for producing an antioxidant effect, wherein the co-crystal has antioxidant properties.

The invention also provides a co-crystal of the invention for producing an anti-inflammatory effect, wherein the co-crystal has anti-inflammatory properties.

The invention also provides a co-crystal of the invention for the prophylactic or therapeutic treatment of cardiovascular disease, wherein the co-crystal treats cardiovascular disease.

The invention also provides a co-crystal of the invention for improving cognitive function, wherein the co-crystal has cognitive enhancing properties

The invention also provides a co-crystal of the invention for the prophylactic or therapeutic treatment of Alzheimer's disease, wherein the co-crystal treats Alzheimer's disease.

The invention also provides the use of a co-crystal of the invention to prepare a medicament for treating cancer in an animal, wherein the co-crystal has anticancer properties.

The invention also provides the use of a co-crystal of the invention to prepare a medicament for producing an antioxidant effect in an animal, wherein the co-crystal has antioxidant properties.

The invention also provides the use of a co-crystal of the invention to prepare a medicament for producing an anti-inflammatory effect in an animal, wherein the co-crystal has anti-inflammatory properties.

The invention also provides the use of a co-crystal of the invention to prepare a medicament for treating cardiovascular disease in an animal, wherein the co-crystal treats cardiovascular disease.

The invention also provides the use of a co-crystal of the invention to prepare a medicament for improving cognitive function in an animal, wherein the co-crystal has cognitive enhancing properties

The invention also provides the use of a co-crystal of the invention to prepare a medicament for treating Alzheimer's disease in an animal, wherein the co-crystal treats Alzheimer's disease.

The invention also provides processes and intermediates disclosed herein that are useful for preparing co-crystals of the invention.

### BRIEF DESCRIPTION OF DRAWINGS

The patent or application file contains at least one drawing executed in color. Copies of this patent or patent application publication with color drawings will be provided by the Office upon request and payment of the necessary fee.

FIG. 1. Shows a single crystal structure of naringenin:succinimide co-crystal with 1:1 stoichiometry.

FIG. 2. Shows a single crystal structure of naringenin:betaine co-crystal methanol solvate in 1:1:1 stoichiometry.

FIG. 3. Shows a single crystal structure of naringenin:tetramethyl pyrazine co-crystal in 1:2 ratio.

FIG. 4. Shows an overlay of diffraction patterns of naringenin (NAR), succinimide, physical mixture and co-crystal.

FIG. 5. Shows an overlay of DSC thermograms of NAR, succinimide, physical mixture and co-crystal.

FIG. 6. Shows an overlay of simulated diffraction pattern from single crystal of co-crystal with experimental pattern.

FIG. 7. Shows powder diffraction patterns overlaid and compared to confirm the formation of three new co-crystal phases in 1:1, 1:2 and 1:1.5 ratios.

FIG. 8. Shows DSC thermograms of NAR, TMP, PM and two new co-crystal phases.

FIG. 9. Shows ¹H NMR spectrum of NAR:TMP co-crystal suggesting 1:1 stoichiometry.

FIG. 10. Shows ¹H NMR spectra of NAR:TMP co-crystal sample weighed in 1:3 ratio indicating the presence of co-crystal in 1:1.5 ratio by comparing the integrated intensities.

FIG. 11. Shows an overlay of powder X-ray diffraction patterns of physical mixture of HES:TMP (bottom trace) and HES:TMP cocrystal (top trace).

FIG. 12. Shows DSC thermograms of HES, TMP, physical mixture of HES:TMP and HES:TMP cocrystal are overlaid, indicating distinct melting point of cocrystal at 178.39° C.

FIG. 13. Shows ¹H NMR spectrum of HES:TMP cocrystal, suggesting 2:1 stoichiometry.

FIG. 14. Shows an overlay of diffraction spectra of quercetin, succinimide, physical mixture and co-crystal.

FIG. 15. Shows DSC thermograms of quercetin, succinimide, physical mixture and co-crystal are overlaid and indicates the three endotherms characteristic of co-crystal phase.

FIG. 16. Shows ¹H NMR spectrum of quercetin:succinimide cocrystal in deuterated DMSO solvent, implying 4:1 stoichiometry.

FIG. 17. Shows an overlay of diffraction spectra of naringenin:aspartame physical mixture and co-crystal phase

FIG. 18. Shows DSC thermograms of naringenin, aspartame, physical mixture and co-crystal are overlaid and indicates the three endotherms characteristic of co-crystal phase.

FIG. 19. Shows FT-IR spectra for naringenin-aspartame physical mixture and co-crystal phase were compared wherein emergence of new peaks and shift in peaks observed for co-crystal phase.

FIG. 20. Shows ¹H NMR spectrum of naringenin:aspartame cocrystal in deuterated DMSO solvent, implying 1:2 stoichiometry.

FIG. 21. Shows powder diffraction patterns of NAR:hexamine system overlaid for the physical mixture and the cocrystal obtained from the ethyl acetate-based slurry.

FIG. 22. Shows an overlay of DSC thermograms of a physical mixture and the cocrystal (NAR:Hexamine).

FIGS. 23A-23C. Show ¹H-NMR spectra of FIG. 23A) naringenin (NAR) FIG. 23B) Hexamine and FIG. 23C) NAR:hexamine, indicating 1:1 stoichiometry.

FIG. 24. Shows an overlay of dissolution profiles of NAR, NAR:succinimide (SUC) physical mixture and NAR:SUC cocrystal.

FIG. 25. Shows the single crystal structure of naringenin:TMP cocrystal with 1:1 stoichiometry (Example 2)

FIG. 26. Shows an overlay of the powder diffraction patterns for four NAR:TMP cocrystals with different stoichiometry 1:1 form I, 1:1 form II, 1:2, and 2:3 (1:1.5)

FIG. 27. Shows ¹H-NMR analysis of NAR:TMP cocrystal form II indicating 1:1 stoichiometry.

FIG. 28. Shows dissolution profiles for NAR concentrations for NAR:TMP cocrystals and physical mixtures in pH 5 acetate buffer in non-sink conditions at room temperature.

FIG. 29. Shows a comparison of relative area under curve (AUC) of different NAR: TMP systems compared to AUC of NAR in pH 5 acetate buffer dissolution studies.

FIG. 30. Shows dissolution profiles for TMP concentrations for NAR:TMP cocrystals and physical mixtures in pH 5 acetate buffer in non-sink conditions at room temperature.

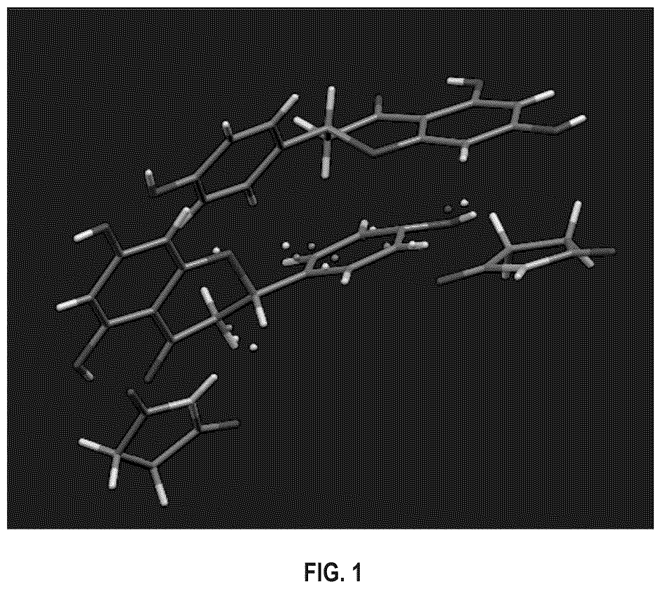

### DETAILED DESCRIPTION

The term “about” means plus or minus 10% of the given value.

The term “flavonoid” is well-known and includes over 5,000 hydroxylated phenolic compounds. The term includes sesquiterpenes with variable phenolic structures. Flavonoids can be classified into flavonols (e.g., quercetin, rutin), flavanones (e.g., naringenin, hesperidin), flavanols (e.g., epicatechin, gallocatechin), flavones (e.g., luteolin, apigenin), and anthocyanins (e.g., pelargonidin, malvidin). In one embodiment, the term includes quercetin, rutin, macluraxanthone, genistein, scopoletin, daidzein, taxifolin, naringenin, abyssinones, rutin, eriodictyol, fisetin, theaflavin, peonidin, diosmetin, tricin, biochanin, hesperidin, epicatechin, kaempferol, luteolin, and apigenin.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention,

The phrase “therapeutically effective amount” or “effective amount” includes but is not limited to an amount of a co-crystal that (i) treats or prevents the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein.

The term “animal” includes mammals, fish, amphibians, reptiles, birds and invertebrates. The term “mammal” includes humans, higher non-human primates, rodents, domestic, cows, horses, pigs, sheep, dogs and cats. In one embodiment, the animal is a mammal. In one embodiment, the animal is a human. The term “patient” as used herein refers to any animal including mammals. In one embodiment, the patient is a mammalian patient. In one embodiment, the patient is a human patient.

The pharmaceutical compositions of the invention can comprise one or more excipients. When used in combination with the pharmaceutical compositions of the invention the term “excipients” refers generally to an additional ingredient that is combined with the co-crystal to provide a corresponding composition. For example, when used in combination with the pharmaceutical compositions of the invention the term “excipients” includes, but is not limited to: carriers, binders, disintegrating agents, lubricants, sweetening agents, flavoring agents, coatings, preservatives, and dyes.

Specific embodiments listed below are for illustration. It is to be understood that two or more embodiments may be combined. It is also to be understood that the embodiments listed herein below (or subsets thereof) can be excluded.

In one embodiment, the co-crystal former is a biologically active molecule.

In one embodiment, the co-crystal former is a biologically inactive.

In one embodiment, the co-crystal former has antioxidant, anti-inflammatory, anti-cancer, neuroprotective, platelet aggregation inhibiting, blood vessel dilating, microcirculation improving, vascular endothelium protecting, metabolism improving, antiapoptosis, anti-fibrosis, or nootropic activity.

In one embodiment, the co-crystal former is selected from the group consisting of succinimide, tetramethyl pyrazine, aspartame, hexamine, and betaine methanol.

In one embodiment, the co-crystal former is succinimide.

In one embodiment, the co-crystal former is tetramethyl pyrazine.

In one embodiment, the co-crystal former is betaine methanol.

In one embodiment, the co-crystal former is aspartame.

In one embodiment, the co-crystal former is hexamine.

In one embodiment, the flavonoid and a co-crystal former are present in a ratio of about 1:1, about 1:1.5, about 1:2, about 1:3, about 1:4, about 1:5, about 2:1, about 3:1, about 4:1, or about 5:1.

In one embodiment, the flavonoid and a co-crystal former are present in a ratio of about 1:1, about 1:1.5, about 1:2, about 2:1, or about 4:1.

In one embodiment, the flavonoid and a co-crystal former are present in a ratio of about 1:1 or about 1:2.

In one embodiment, the flavonoid is hesperetin, naringenin, or quercetin.

In one embodiment, the flavonoid is hesperetin.

In one embodiment, the flavonoid is naringenin.

In one embodiment, the flavonoid is quercetin.

In one embodiment, the co-crystal comprises the flavonoid naringenin and the co-crystal former succinimide in a ratio of about 1:1.

In one embodiment, the co-crystal comprises the flavonoid naringenin and the co-crystal former tetramethyl pyrazine.

In one embodiment, the co-crystal comprises the flavonoid naringenin and the co-crystal former tetramethyl pyrazine in a ratio of about 1:1.

In one embodiment, the co-crystal comprises the flavonoid naringenin and the co-crystal former tetramethyl pyrazine in a ratio of about 1:1.5.

In one embodiment, the co-crystal comprises the flavonoid naringenin and the co-crystal former tetramethyl pyrazine in a ratio of about 1:2.

In one embodiment, the co-crystal comprises the flavonoid naringenin and the co-crystal former betaine methanol.

In one embodiment, the co-crystal comprises the flavonoid naringenin and the co-crystal former betaine methanol in a ratio of about 1:1.

In one embodiment, the co-crystal comprises the flavonoid hesperetin and the co-crystal former tetramethyl pyrazine.

In one embodiment, the co-crystal comprises the flavonoid hesperetin and the co-crystal former tetramethyl pyrazine in a ratio of about 2:1.

In one embodiment, the co-crystal comprises the flavonoid quercetin and the co-crystal former succinimide.

In one embodiment, the co-crystal comprises the flavonoid quercetin and the co-crystal former succinimide in a ratio of about 4:1.

In one embodiment, the co-crystal comprises the flavonoid naringenin and the co-crystal former aspartame.

In one embodiment, the co-crystal comprises the flavonoid naringenin and the co-crystal former aspartame in a ratio of about 1:2.

In one embodiment, the co-crystal comprises the flavonoid naringenin and the co-crystal former hexamine in a ratio of about 1:1.

The co-crystals can be formulated as pharmaceutical compositions and administered to a mammalian host, such as a human patient in a variety of forms adapted to the chosen route of administration.

Thus, the co-crystals may be systemically administered, e.g., orally, in combination with a pharmaceutically acceptable vehicle such as an inert diluent or an assimilable edible carrier. They may be enclosed in hard- or soft-shell gelatin capsules, may be compressed into tablets, or may be incorporated directly with the food of the patient's diet. For oral therapeutic administration, the active co-crystal may be combined with one or more excipients and used in the form of ingestible tablets, buccal tablets, troches, capsules, elixirs, suspensions, syrups, wafers, and the like. Such compositions and preparations should contain at least 0.1% of active co-crystal. The percentage of the compositions and preparations may, of course, be varied and may conveniently be between about 2 to about 60% of the weight of a given unit dosage form. The amount of active co-crystal in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. Of course, any material used in preparing any unit dosage form should be pharmaceutically acceptable and substantially non-toxic in the amounts employed. In addition, the active co-crystal may be incorporated into sustained-release preparations and devices.

For topical administration, the co-crystals may be applied in pure form, i.e., when they are liquids. However, it will generally be desirable to administer them to the skin as compositions or formulations, in combination with a dermatologically acceptable carrier, which may be a solid.

Useful solid carriers include finely divided solids such as talc, clay, microcrystalline cellulose, silica, alumina and the like. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the co-crystals of formula (I) to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the co-crystals can be determined by comparing their in vitro activity, and in vivo activity in animal models. Methods for the extrapolation of effective dosages in mice, and other animals, to humans are known to the art; for example, see U.S. Pat. No. 4,938,949.

The amount of the co-crystal required for use in treatment will vary not only with the particular co-crystal selected but also with the route of administration, the nature of the condition being treated and the age and condition of the patient and will be ultimately at the discretion of the attendant physician or clinician.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

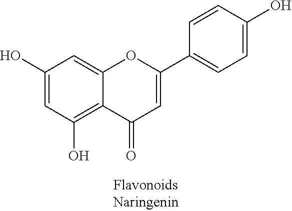 [SMILES: O=c1cc(-c2ccc(O)cc2)oc2cc(O)cc(O)c12]

Naringenin, a citrus flavonoid that possesses various biological activities, has emerged as a potential therapeutic agent for the management of a variety of diseases. Studies using cell culture system have shown that naringenin can inhibit inflammatory response in diverse cell types. Moreover, research using various animal models has further demonstrated therapeutic potentials of naringenin in the treatment of several inflammation-related disorders, such as sepsis, fulminant hepatitis, fibrosis and cancer. The mechanism of action of naringenin is not completely understood but recent mechanistic studies revealed that naringenin suppresses inflammatory cytokine production through both transcriptional and post-transcriptional mechanisms. Surprisingly, naringenin not only inhibits cytokine mRNA expression but also promotes lysosome-dependent cytokine protein degradation. This unique property of naringenin stands in sharp contrast with some widely studied natural products such as apigenin and curcumin, which regulate cytokine production essentially at the transcriptional level. Therefore, naringenin may provide modality for the development of novel anti-inflammatory agent. Several biological activities have been ascribed to this phytochemical, among them antioxidant, antitumor, antiviral, antibacterial, anti-inflammatory, antiadipogenic and cardioprotective effects. Nonetheless, most of the data reported have been obtained from in vitro or in vivo studies. Although some clinical studies have also been performed, the main focus is on naringenin bioavailability and cardioprotective action. In addition, these studies were done in compromised patients (i.e., hypercholesterolemic and overweight), with a dosage ranging between 600 and 800 μM/day, whereas the effect on healthy volunteers is still debatable. In fact, naringenin ability to improve endothelial function has been well-established. Indeed, the currently available data are very promising, but further research on pharmacokinetic and pharmacodynamic aspects is encouraged to improve both available production and delivery methods and to achieve feasible naringenin-based clinical formulations. See Kiran S, et al., Journal of Pharmacognosy and Phytochemistry. 2017; 6(5):2778-83; Patel K, Singh G K, Patel D K. A review on pharmacological and analytical aspects of naringenin. Chinese journal of integrative medicine. 2018; 24:551-60; and Salehi B, et al., Pharmaceuticals. 2019; 12(1):11.

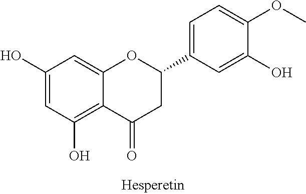 [SMILES: COc1ccc([C@@H]2CC(=O)c3c(O)cc(O)cc3O2)cc1O]

Hesperetin, an important bioactive compound in Chinese traditional medicine, has antioxidant and anticarcinogenic properties. Hesperetin is found in abundance in orange and grape juices (200-590 mg L-1) consumed in the daily diet. Both hesperidin and its aglycone hesperetin have shown various biological activities. For example, hesperetin possesses vitamin-like activity and can decrease capillary permeability (vitamin P), leakiness, and fragility. Currently, it has been indicated that hesperetin confers marked antioxidant, anti-inflammatory, and neuroprotective effects in different models of neurodegeneration. See Poorly GPDG. North American NeuroEndocrine Tumor Society, Sep. 29-Oct. 1, 2016, Jackson Hole, Wyoming. Pancreas. 2017; 46(3); Khan A, et al., Antioxidants. 2020; 9(7):609; Cho J. Archives of pharmacal research. 2006; 29:699-706; Roohbakhsh A, et al., Life sciences. 2015; 124:64-74; and Erlund I. Nutrition research. 2004; 24(10):851-74.

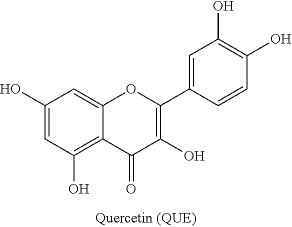 [SMILES: O=c1c(O)c(-c2ccc(O)c(O)c2)oc2cc(O)cc(O)c12]

Quercetin, a flavonoid found in fruits and vegetables, has unique biological properties that may improve mental/physical performance and reduce infection risk. These properties form the basis for potential benefits to overall health and disease resistance, including anti-carcinogenic, anti-inflammatory, antiviral, antioxidant, and psychostimulant activities, as well as the ability to inhibit lipid peroxidation, platelet aggregation and capillary permeability, and to stimulate mitochondrial biogenesis. The first investigation on the pharmacokinetics of quercetin in humans suggested very poor oral bioavailability after a single oral dose (˜2%). The estimated absorption of quercetin glucoside, the naturally occurring form of quercetin, ranges from 3% to 17% in healthy individuals receiving 100 mg. The relatively low bioavailability of quercetin may be attributed to its low absorption, extensive metabolism and/or rapid elimination. See Zhang M, et al., editors. Antioxidant properties of quercetin. Oxygen transport to tissue XXXII; 2011: Springer; Boots A W, et al., European journal of pharmacology. 2008; 585(2-3):325-37; and Li Y, et al., Nutrients. 2016; 8(3):167.

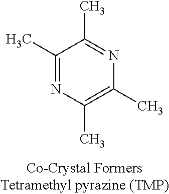

Recent pharmacological studies have shown that TMP has been shown to have various pharmacological activities, including inhibiting platelet aggregation, dilating blood vessels, improving microcirculation, protecting vascular endothelium, improving metabolism, anti-inflammation, antiapoptosis, anti-oxidation, anti-fibrosis, and other conditions. TMP has stable pharmacological activity with few side effects and can penetrate the blood-brain barrier Blood-brain barrier (BBB). It is widely used to treat cardiovascular disease, nervous system diseases, tumors, digestive system diseases, and other conditions. Zhang et al. demonstrated that TMP could penetrate the BBB well without apparent toxic or side effects, but the mechanism of TMP remains unknown. See Xu T, et al., Pharmacological Research—Modern Chinese Medicine. 2022: 100171; Huang X, et al., Frontiers in Cell and Developmental Biology. 2021; 9:632843; Lin, J., et al., Biomedicine and Pharmacotherapy, 2022, 150, 113005; and Weng G, et al., Drug Design, Development and Therapy. 2021: 2385-99.

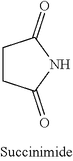

Succinimides have been observed to play a significant role in therapeutic strategies. However, the use of succinimide derivatives is supposed to be a virtuous way to improve metabolic stability and pharmacokinetic properties. Various nitrogen-containing derivatives use succinimide derivatives as the building blocks. Reagents are required for the irregular addition. Succinimides are the well-known class of compounds possessing anti-Alzheimer potential through dual inhibitory pathways. They follow the cholinesterase inhibition at one side and behave as antioxidants on the other. These classes of compounds also have been reported for other pharmacological activities. See Alshehri O M, et al., Evidence-Based Complementary and Alternative Medicine. 2022; 2022; Sadiq A, et al., Chemistry Central Journal. 2015; 9:1-9; Zhao, Z., et al., Bioorganic Chemistry, 2021, 108, 104577; and Waheed B, et al., Evidence-Based Complementary and Alternative Medicine. 2022; 2022.

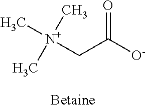

Betaine, that is trimethylglycine, is an endogenous catabolite of choline in mammals including humans. As a natural compound, betaine can be obtained from vegetables and marine products. As three active methyl groups are contained in its structure, betaine is presumed to serve as an effective methyl donor for Hcy remethylation. Epidemiology and accumulating clinical investigations have shown that the elevated plasma homocysteine (Hcy) has a positive correlation with the occurrence of AD, and thus hyperhomocysteinemia (Hhcy) has been proposed to be a strong and independent risk factor of AD In fact, betaine supplementation can effectively promote the metabolism of Hcy and reduce the plasma Hcy levels in healthy subjects. Previous clinical studies suggested that betaine supplementation is effective in reducing plasma Hcy levels of patients with MTHFR deficiency and/or genotype mutation. It has also been successfully administered to coronary atherosclerosis, fatty liver, and hyperlipidemia in clinic. See Chai G S, et al., Journal of neurochemistry. 2013; 124(3):388-96; Ramakrishna T, et al., Current Science. 1998: 1153-6; Kaur, S., et al., Plant Archives, 2019, 19 (Supplement 2), 1021-1034; and Sun S, et al., Scientific reports. 2016; 6(1):35547.

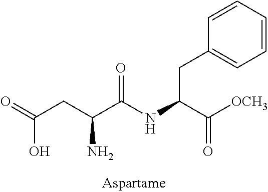 [SMILES: COC(=O)[C@H](Cc1ccccc1)NC(=O)[C@@H](N)CC(=O)O]

Aspartame (C₁₄H₁₈N₂O₅) is a common sugar-free sweetener known commercially by the brand names of Equal or NutraSweet. It is used in pharmaceutical products, often as a sugar replacement in chewable tablets and sugar-free liquids. The FDA approved the use of aspartame in food products in 1981. It is an artificial sweetener, often consumed as a sugar replacement in various foods and beverages. Chemically, aspartame is a methyl ester of phenylalanine. The attractiveness of aspartame as a sweetener is since it is about 200 times sweeter than sugar, while its calorific value, at the concentrations giving the impression of sweetness, is almost zero. However, the taste of aspartame is not identical to that of regular sugar: the flavor takes longer to appear, and typically has an aftertaste. According to the FDA, the acceptable daily intake of aspartame for humans is 40 mg/kg bodyweight in Europe and 50 mg/kg bodyweight in the United States for both adults and children (Lindley M G. New developments in low-calorie sweeteners. Low-Calories Sweeteners: Present and Future. 1999; 85:44-51; and Drugs C. Inactive. Ingredients in Pharmaceutical Products: Update (Subject Review), Pediatrics. 1997; 99:268-78).

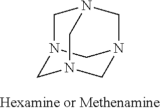

Methenamine is a heterocyclic organic compound with a cage-like structure similar to adamantane. It has a role as an antibacterial drug. It is a polycyclic cage, a polyazaalkane and a tetramine. Methenamine is a urinary tract antiseptic and antibacterial drug used for the prophylaxis and treatment of frequently recurring urinary tract infections requiring a long-term therapy. Methenamine has not been linked to serum enzyme elevations or to instances of clinically apparent acute liver injury. The researchers say methenamine could be an alternative to antibiotics for preventing frequent urinary tract infections, thereby reducing the use of antibiotics (evidence.nihr.ac.uk/alert/methenamine-as-good-as-antibiotics-preventing-urinary-tract-infections/; medlineplus.gov/druginfo/meds/a682296.html; and go.drugbank.com/drugs/DB06799)

The invention will now be illustrated by the following non-limiting Examples.

### EXAMPLES

### Co-Crystal Preparation Process

### Liquid Assisted Grinding

Stoichiometric ratios of flavonoid and coformer (1:1 or 1:2 or 1:3) were loaded into a stainless-steel grinding jar with total weight of 200 mg that contained two grinding balls (Φ=10 mm, stainless steel). An accurate amount of solvent (0.25 ml ug−1 or 1 ml ug−1) was added to the jar to facilitate potential cocrystal formation through liquid assisted grinding. Powders were milled for 30 minutes using an FTS vibratory mill (located in the Gillan laboratory, University of Iowa Chemistry) at 1200 rpm. The obtained products were dried at 70° C. for 24 hours to remove any residual solvent, followed by storage at room temperature in a desiccator.

### Solution Mediated Slurry Process

Solvent-based slurries were also used for cocrystal preparation. Molar ratios of flavonoid:coformer were suspended in a beaker with 2-3 mL of solvent and slurred at 250 rpm using a magnetic stir plate for 72 hours at room temperature. The suspension was filtered using a 0.22-micron filter, and the wet solid was dried at 70° C. for 24 hours to remove any residual water. All materials were stored in the desiccator to minimize the effects of humidity.

### Single Crystal Preparation

Single-crystal growth for new co-crystal was prepared using slow evaporation or heat cool method. For slow evaporation method, equimolar saturated solutions of drug and co-formers were prepared in vial and covered loosely with lid. Solutions were kept in a vibration free zone to evaporate until either complete drying or single crystals suitable for XRD were visible. In case of heat cool method, molar ratios of drug: co-former were added to the solvent to prepare the suspension and then was heated until it becomes a clear solution. The solution was cooled gradually at room temperature to generate supersaturation with respect to co-crystal and get a good quality of crystals.

### Physical and Structural Characterization

### Powder X-Ray Diffraction (PXRD)

Powder X-ray diffraction was used to confirm the formation of new phase, i.e., co-crystal and guide to understand purity of samples. Powders were ground using a mortar and pestle to minimize particle size differences. Powder patterns were collected using a Siemens D8 diffractometer from Bruker located in the University of Iowa, Chemistry Department. X-rays generated from a Cu source (Cu Kα λ=1.5418 Å) were used as an incident source. The powders were loaded into a sample holder, and room-temperature powder patterns were collected at 2θ values ranging from 2.5° to 40° with a step size of 0.02° and a dwell time of 0.75 seconds. The diffraction spectra were analyzed using MDI JADE software.

### Thermal Analysis

As a complementary method of phase analysis, each material was evaluated using differential scanning calorimetry (DSC). DSC scans were performed using a TA Instruments Q20, calibrated with an indium standard. Approximately 1-5 mg of powders were used for samples. The aluminum pan was crimped with a lid and heated from ˜25 to 300° C. (about 20° above the reported melting point of either drug/coformer), at a rate of 20° C./minute under a nitrogen purge of 40 mL/min. Obtained thermograms were analyzed using the Universal Analysis software package provided from TA Instruments.

### Single Crystal Structural Analysis

A single crystal of co-crystal obtained using above-mentioned methods used for the X-ray crystallographic analysis. Data were collected on a Bruker D8 VENTURE DUO diffractometer equipped with a IμS 3.0 microfocus source operated at 75 W (50 kV, 1.5 mA) to generate Mo Kα radiation (λ=0.71073 Å) and a PHOTON III detector. Mo Kα radiation (λ=0.71073 Å) was used as the incident source, with total exposure time ranging from 3 hours to 15 hours. The crystal and a small amount of the oil were collected on a MiTeGen 100 micron MicroLoop and transferred to the instrument where it was placed under a cold nitrogen stream (Oxford 800 series) maintained at 100K throughout the duration of the experiment. A unit cell collection was then carried out. After it was determined that the unit cell was not present in the CCDC database a data collection strategy was calculated by APEX4. Intensity data were corrected for Lorentz, polarization, and background effects using the APEX4. A semi-empirical correction for adsorption was applied using SADABS1. The program SHELXT2 was used for the initial structure solution and SHELXL3 was used for the refinement of the structure. Both programs were utilized within the OLEX2 software. The .cif files obtained from single crystal X-ray diffraction were used to plot out the crystal structure using Mercury 3.8, a software provided by the CSD.

### Proton Nuclear Magnetic Resonance (¹H NMR)

For cocrystals that did not yield single crystals, proton NMR was performed to confirm the stoichiometry. All measurements were obtained at 500 MHz conducted on Bruker AVIII instrument (Billerica, MA USA) operating at a proton frequency of 500 MHz and a 1.7 mm PATXI, Z gradient probe under the control of Topspin software. The sample temperature was maintained at 298K throughout. Spectral windows of 10 ppm were used for both domains. All free induction decays were acquired at room temperature (25° C.) under steady state conditions with non-spinning samples using standard Bruker parameter sets, and 32 scans were collected per sample. Clear solutions of the samples were prepared at a concentration of 1-5 mg/mL in deuterated methanol or DMSO in 7-inch (5 mm) NMR glass tubes with a total of 600 μl in each tube (n=3).

### Co-Crystal Preparation

### Example 1. Preparation of Naringenin-Succinimide Co-Crystal

The liquid assisted grinding and slurry methods produced a pure co-crystal phase. Co-crystal was obtained with all of the following solvents: ethyl acetate, methanol, ethanol and acetonitrile. Also, co-crystal phase was generated with use of either 0.25 ml/ug or 1 ml/ug solvent amount. In case of slurry experiments, nature of solvent did not influence the formation and purity of the co-crystal. Ethyl acetate, isopropyl alcohol, methanol solvent resulted in the same co-crystal phase. The co-crystal formation was instantaneous with slurry method, highest rate with methanol. The formation of co-crystal was confirmed by PXRD, DSC and solution NMR. The diffraction pattern shows characteristic peaks at 5.1°, 10.1° and 15.1° and single endotherm was observed at 210° C. for pure co-crystal phase.

### Example 2. Preparation of Naringenin-Tetramethyl Pyrazine Co-Crystal

Both LAG and slurry methods resulted in new co-crystal phases. The slurry method was optimized to produce the pure phase of the co-crystal. Different solvents such as methanol, ethanol, IPA and ethyl acetate were tried to get rid of the starting materials. The phase obtained using the ethyl acetate was pure and without any trace of starting materials. The generation of co-crystal was not sensitive to the nature of solvent however the purity of the co-crystal was dependent on the solvent and solubility of individual components. Due to the presence of two hydrogen accepting nitrogen groups in the co-former, multiple stoichiometry ratios were tried to generate the higher-order co-crystals. Three slurry samples prepared using ethyl acetate in 1:1 and 1:3 ratios showed distinguishable peaks compared to physical mixture, confirming the formation of three different co-crystal phases of NAR:TMP. The fourth cocrystal of NAR:TMP was obtained through LAG method.

DSC analysis supports the formation of two co-crystals as single endotherm was observed for sample in 1:1 ratio at 169.97° C. with 96.74 J/g ΔH_{f} whereas two endotherms for sample weighed in 1:3 ratio, first one at 146.78° C. with 42.32 J/g ΔH_{f} and another one 165.69° C. with 61.91 J/g ΔH_{f}. The stoichiometry of the drug:co-former in co-crystal was determined using solution state 1H NMR. The integrated intensities corresponding to one proton of NAR at 11.8 ppm and twelve protons at 2.2 ppm for TMP respectively, were ratioed in co-crystal sample and used to determine the stoichiometry of drug:co-former. Three co-crystal phases suggested to present in 1:1 (two forms) and 1:1.5 ratio. Apart from these samples, single crystal analysis of the NAR:TMP co-crystal, prepared with heat-cool method, using X-ray diffraction confirmed the formation of co-crystal of NAR:TMP in 1:2 and 1:1 ratio. Overall, four new co-crystal phases of NAR:TMP were generated in 1:1 (two forms), 1:1.5 and 1:2 ratios.

### Example 3. Preparation of Naringenin-Betaine Co-Crystal Methanol Solvate

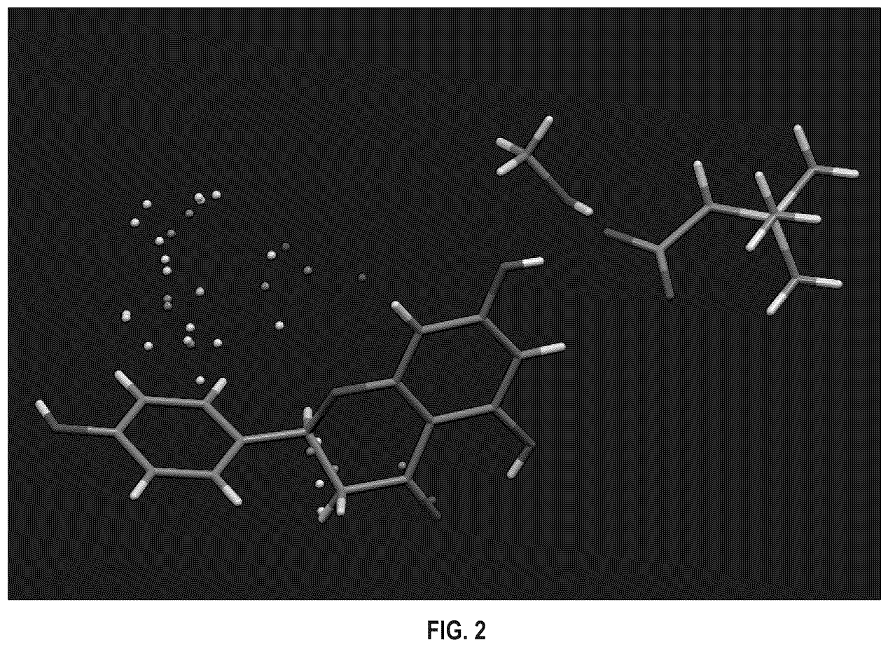

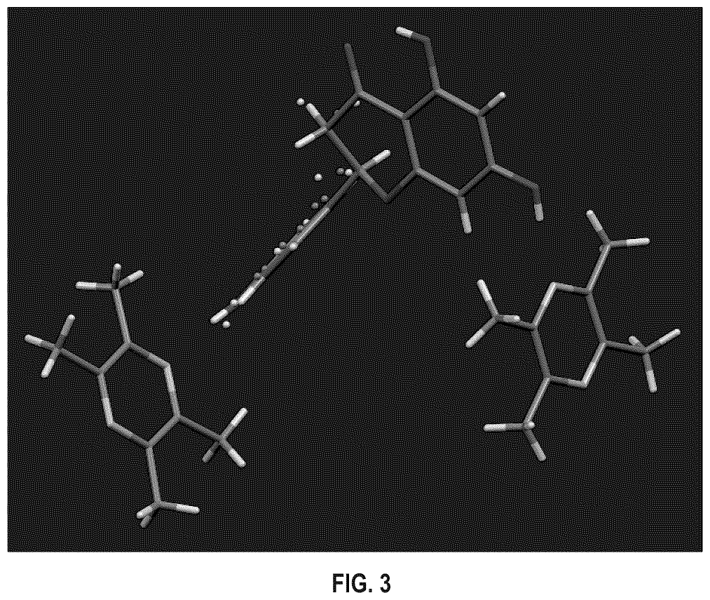

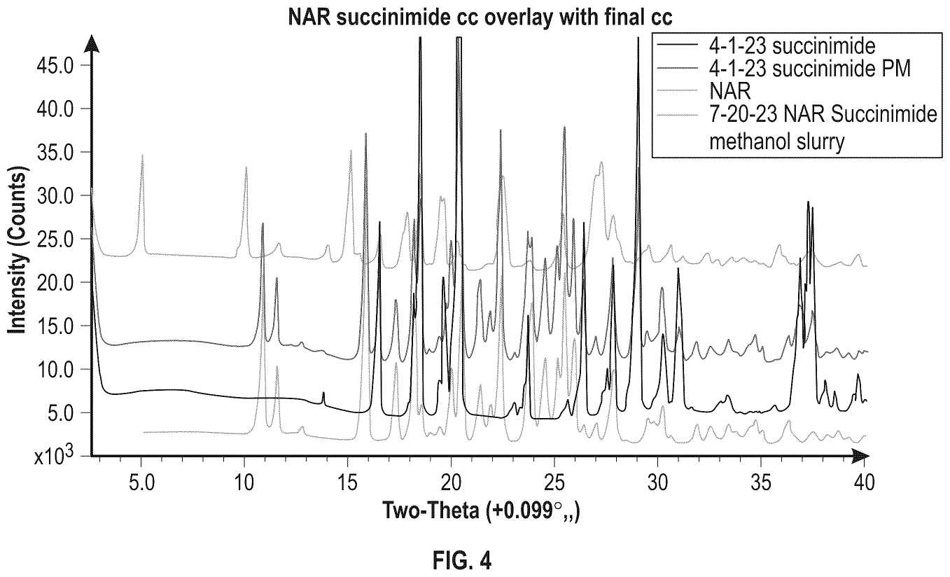

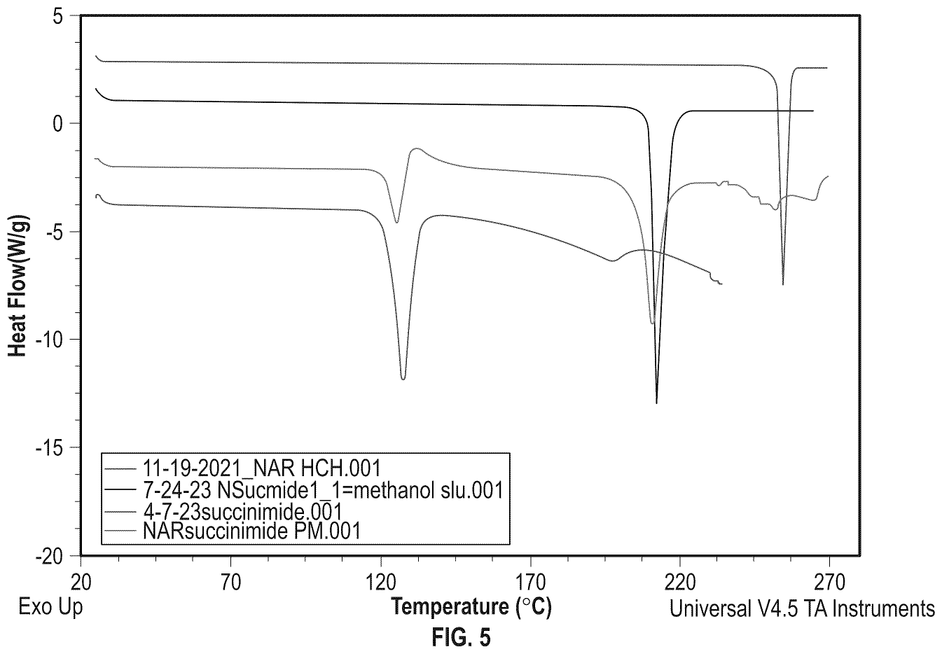

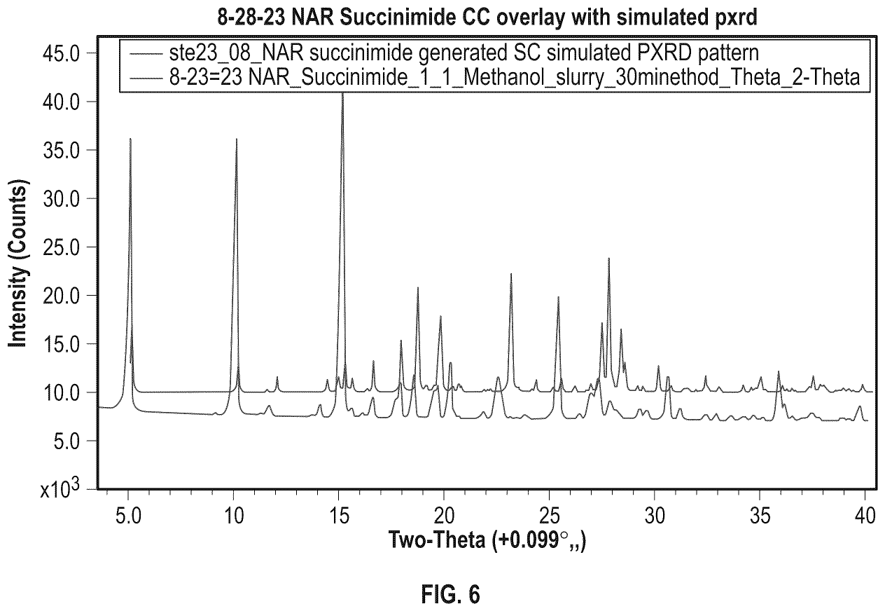

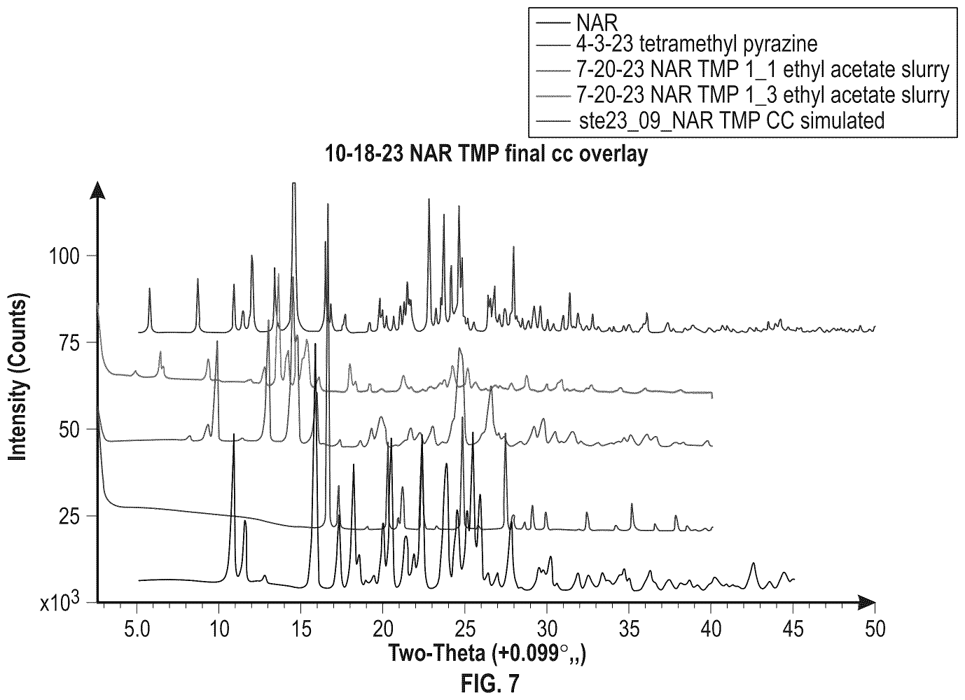

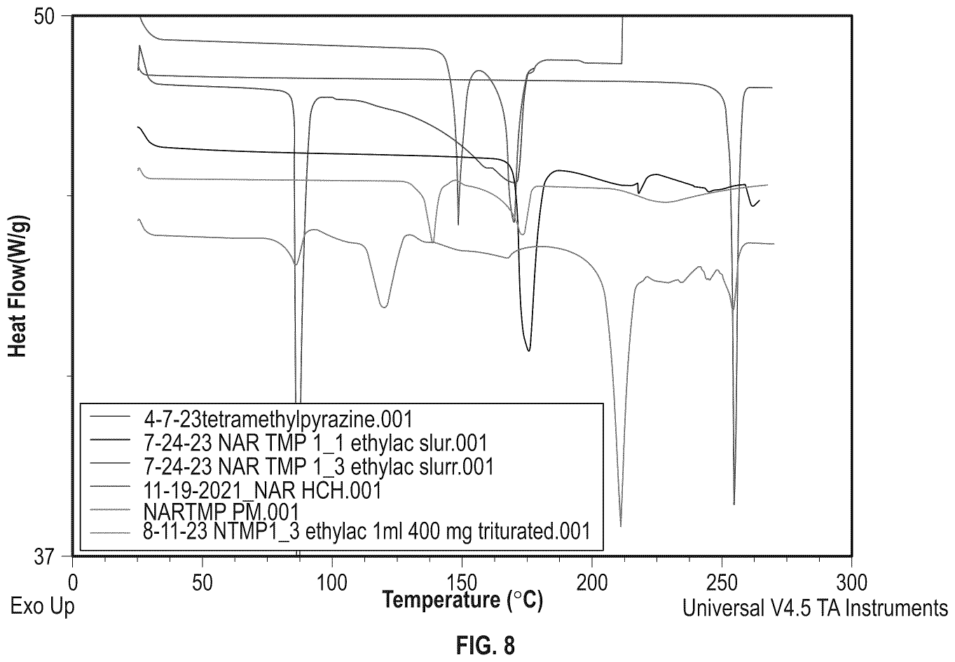

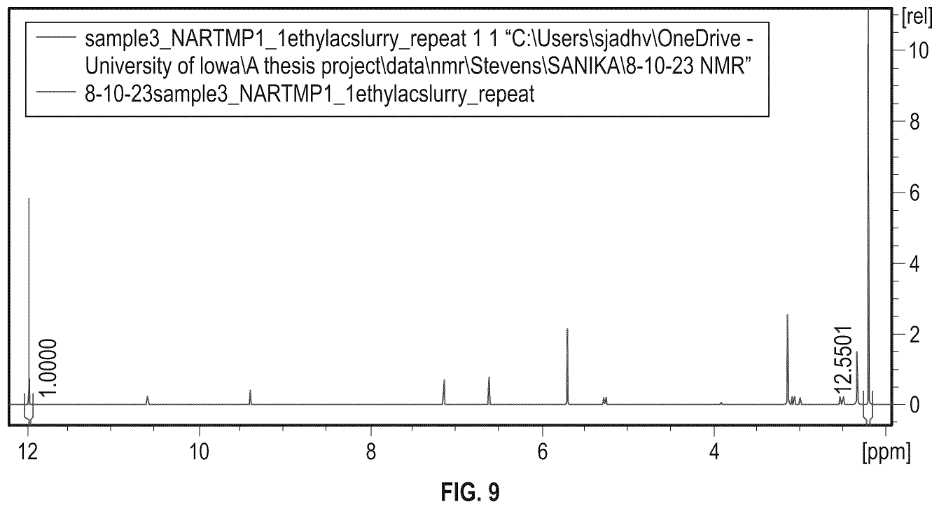

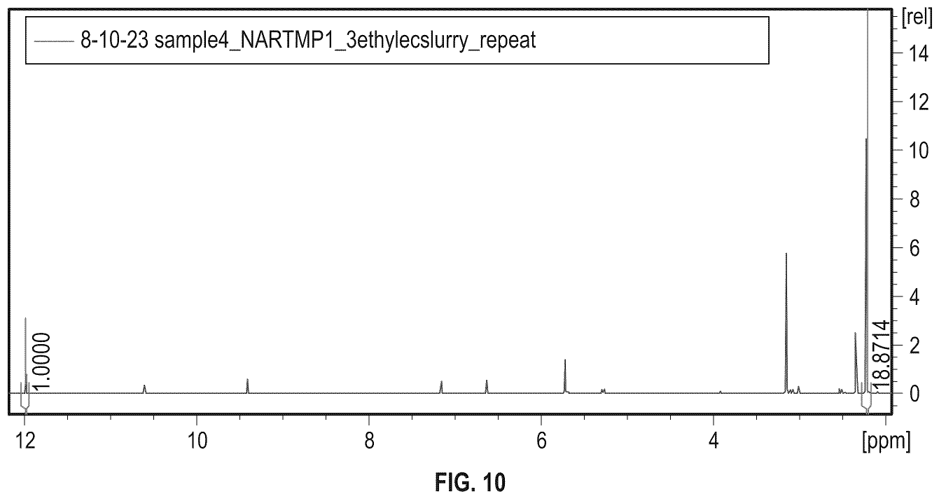

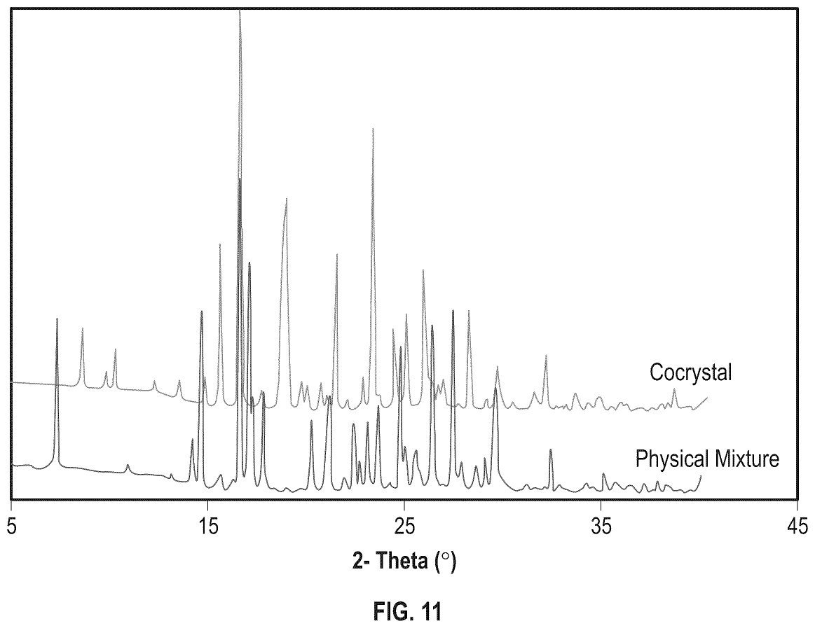

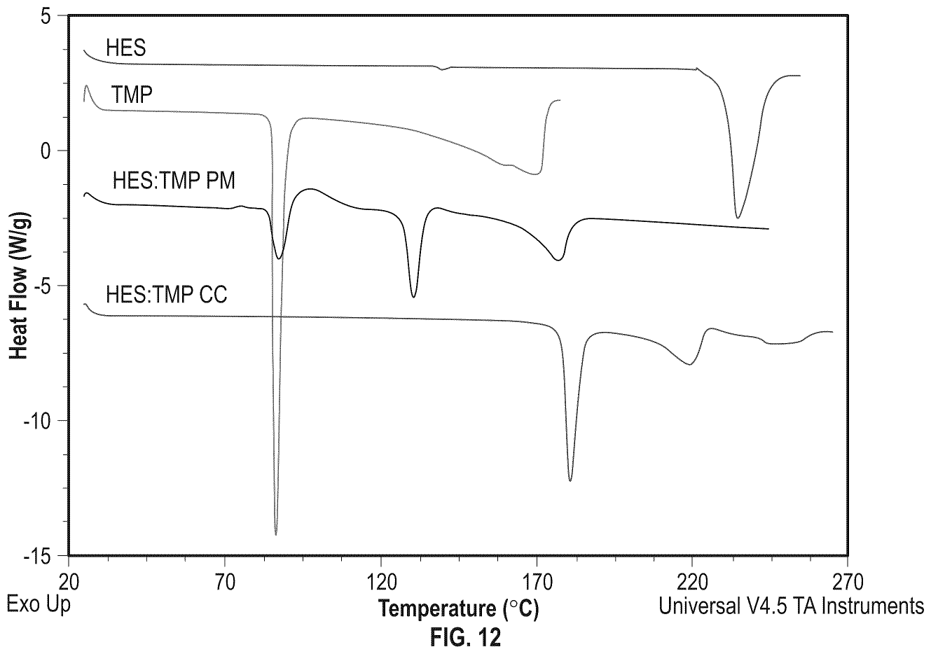

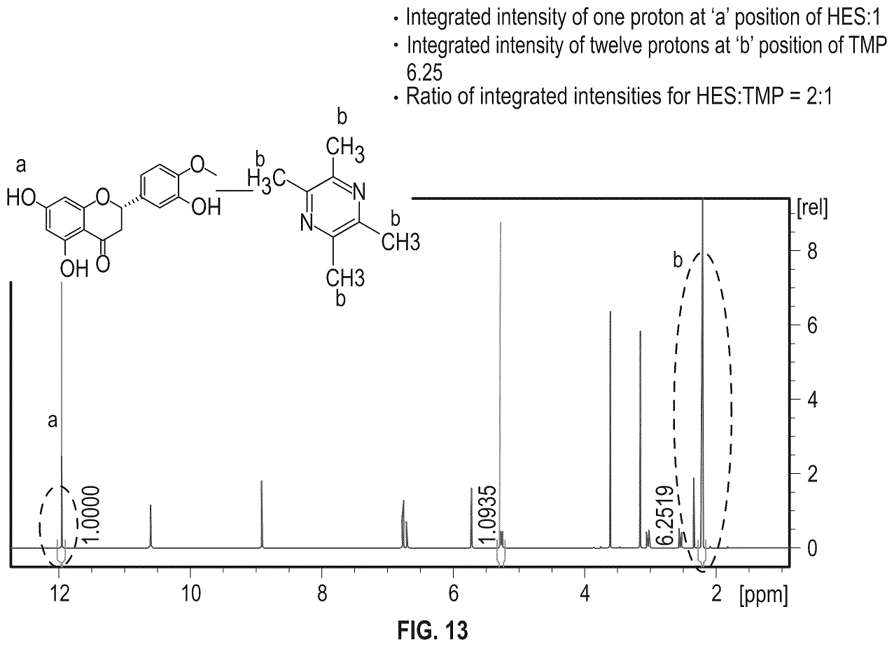

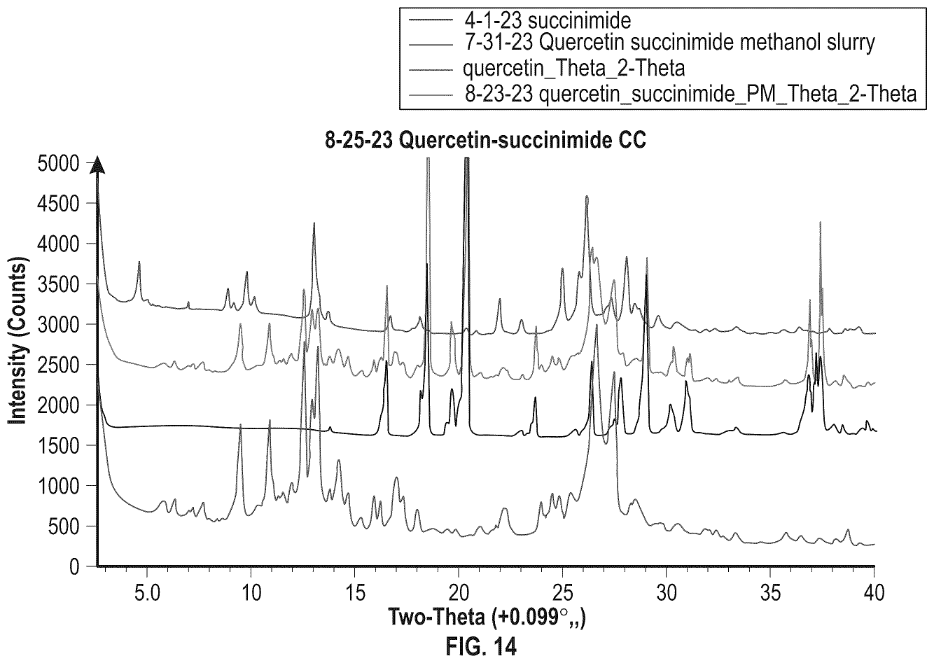

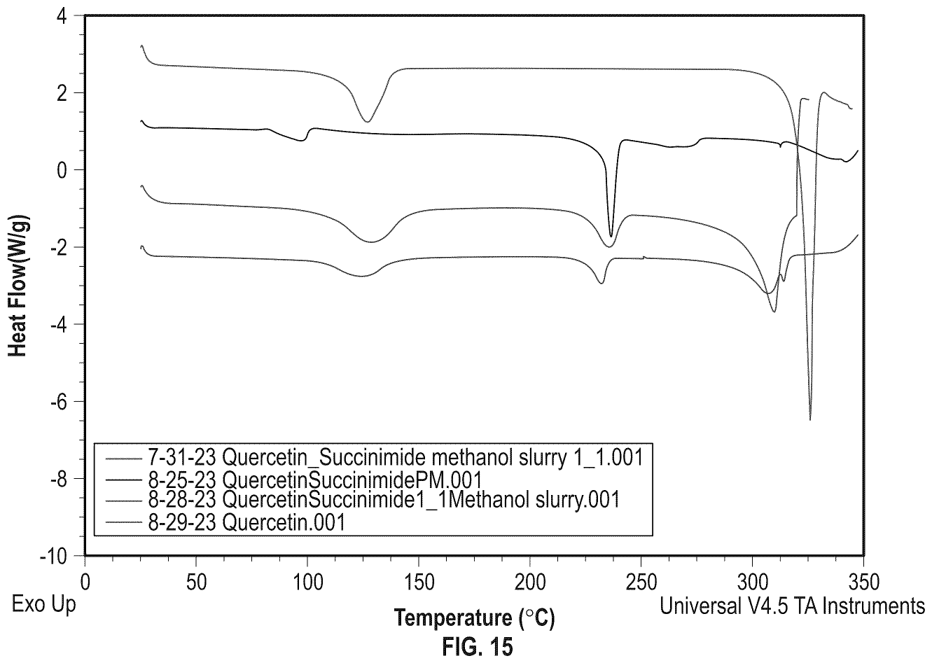

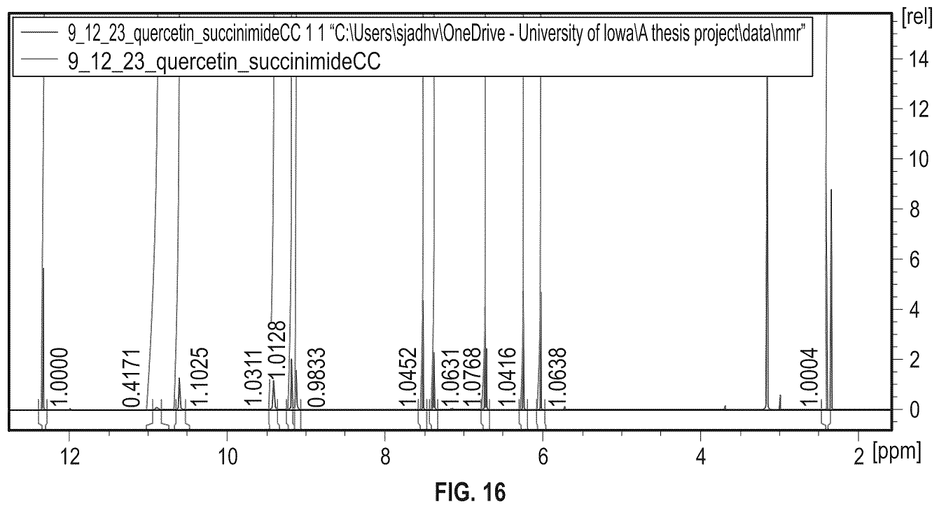

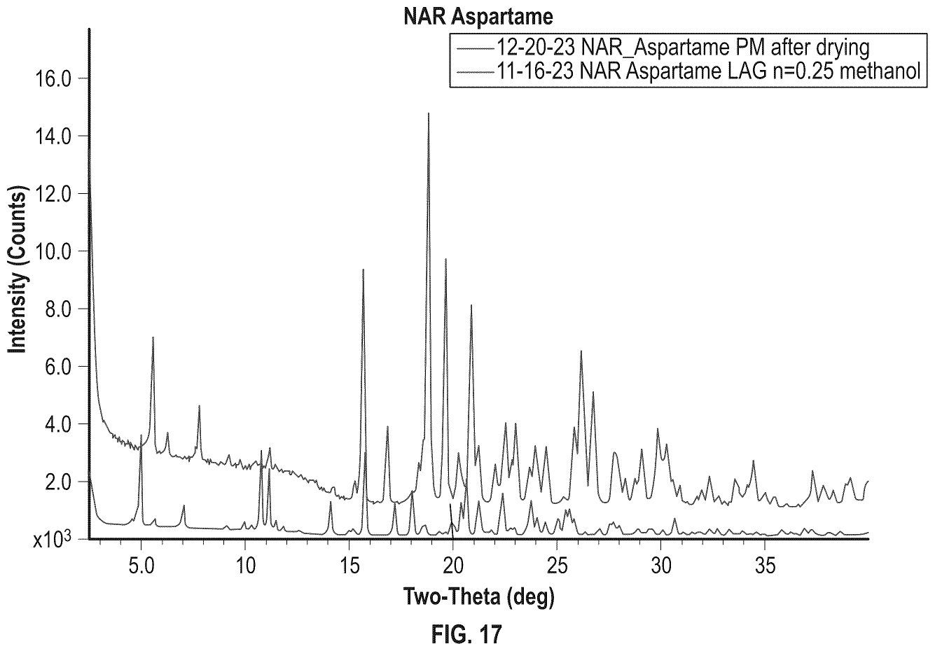

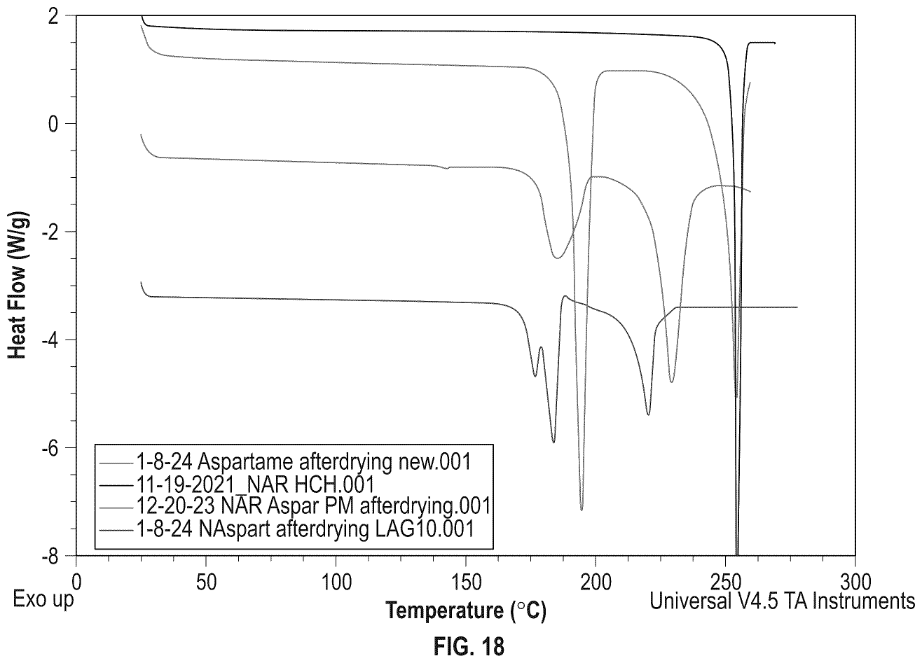

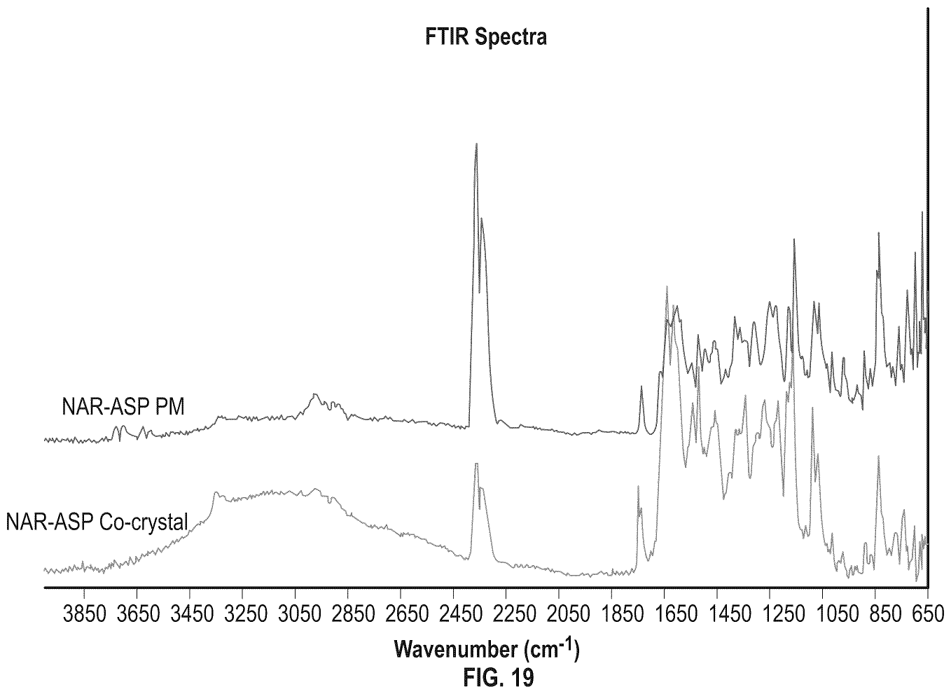

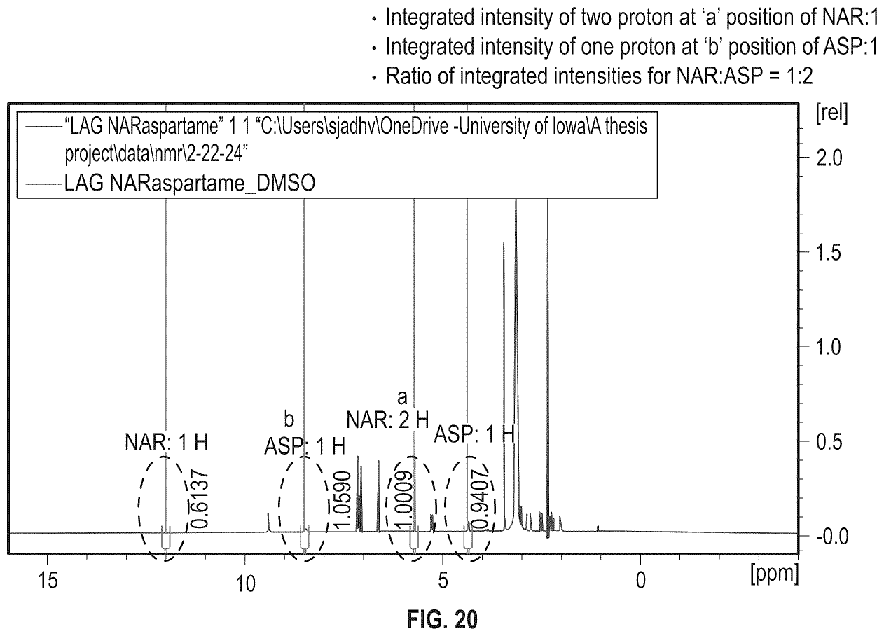

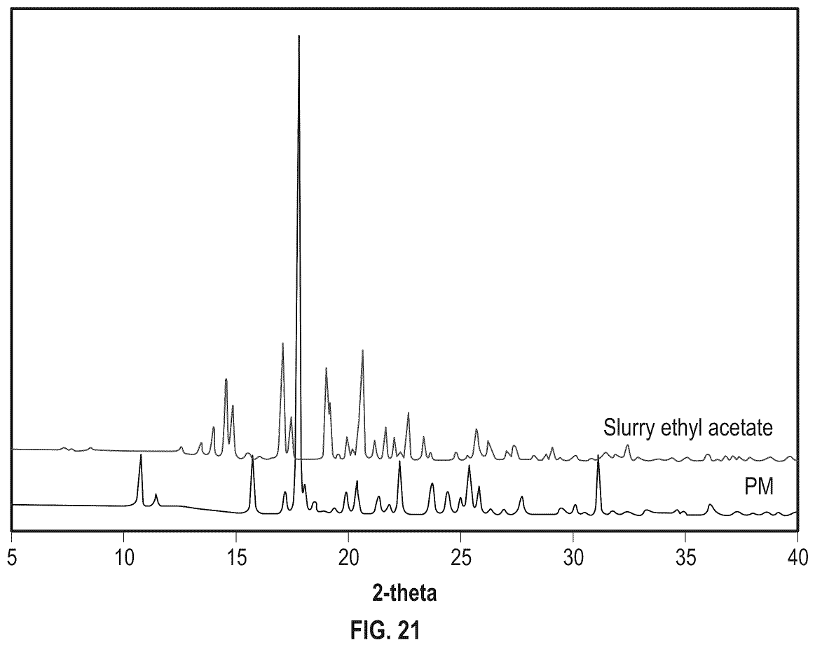

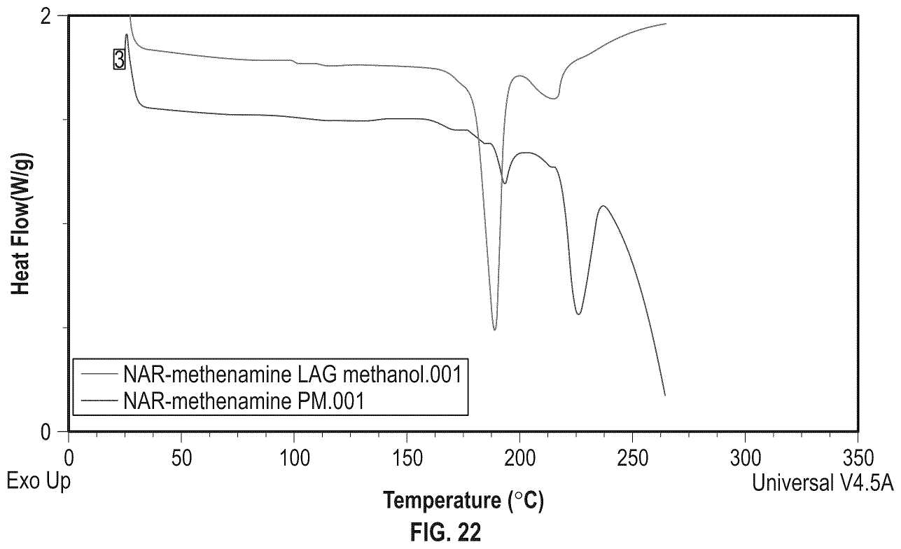

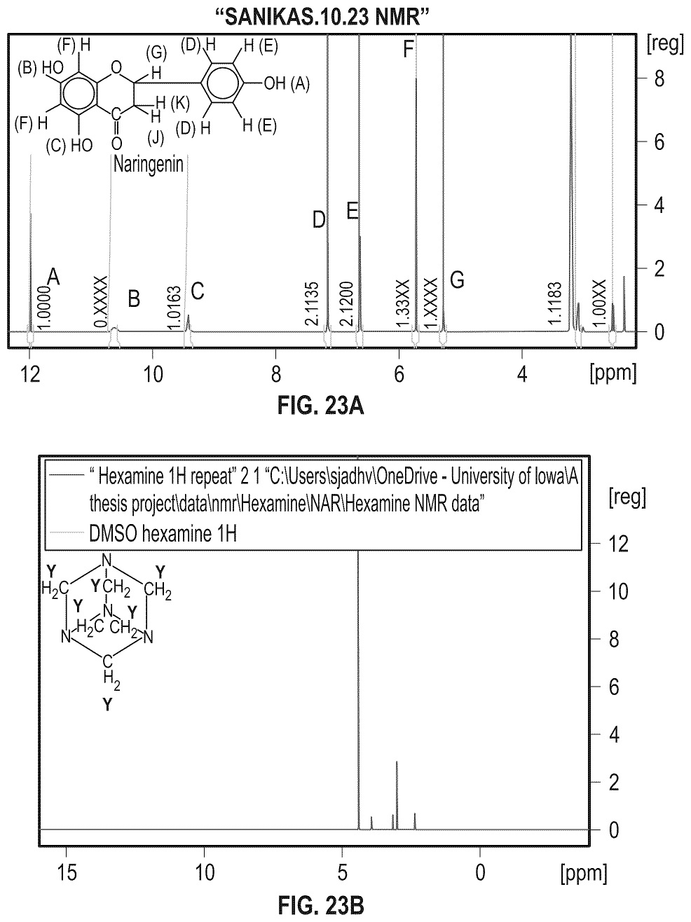

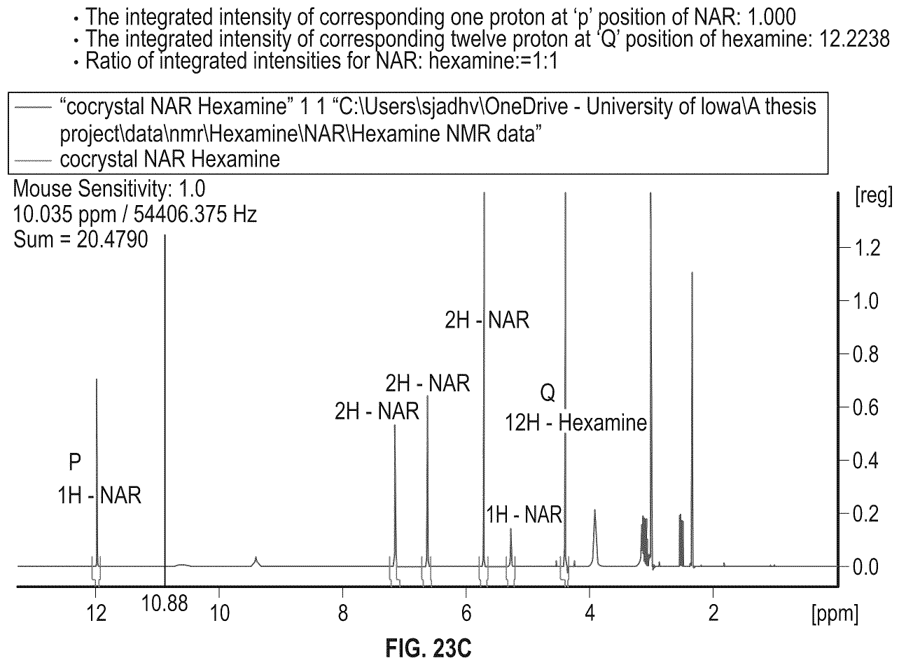

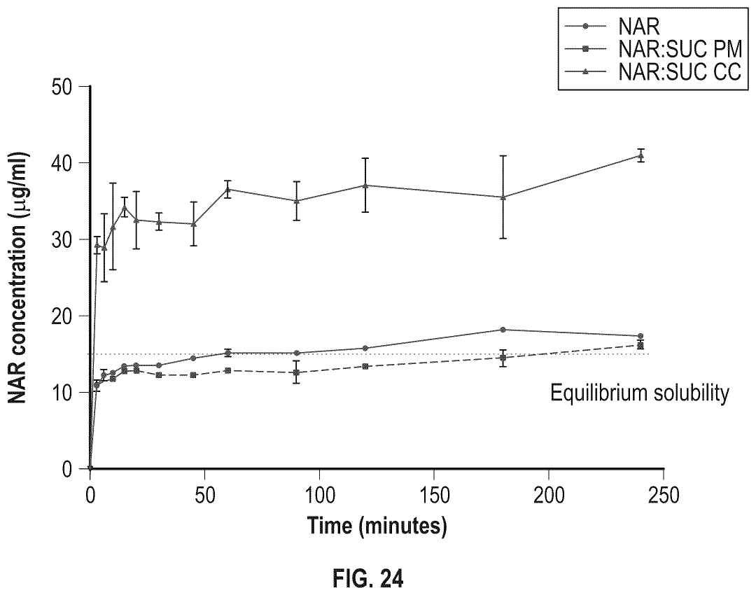

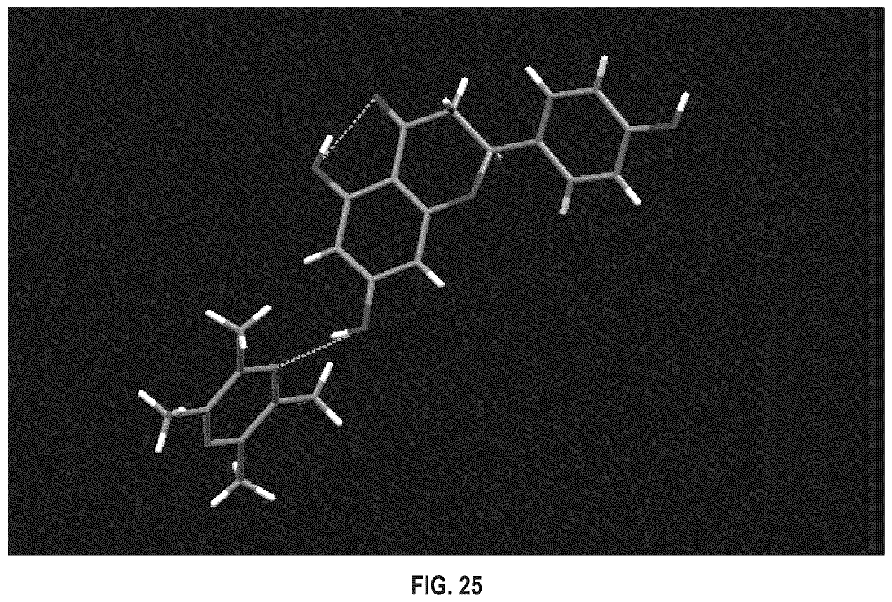

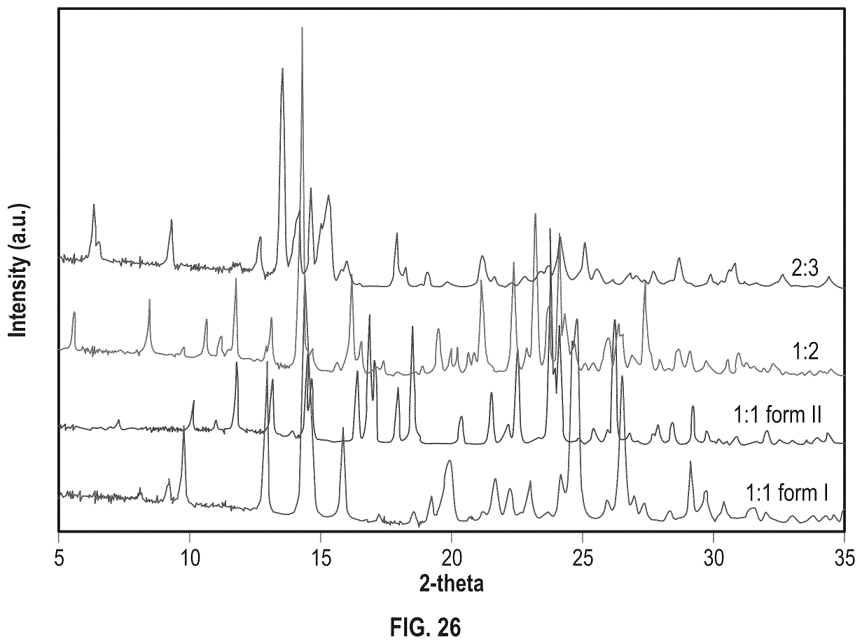

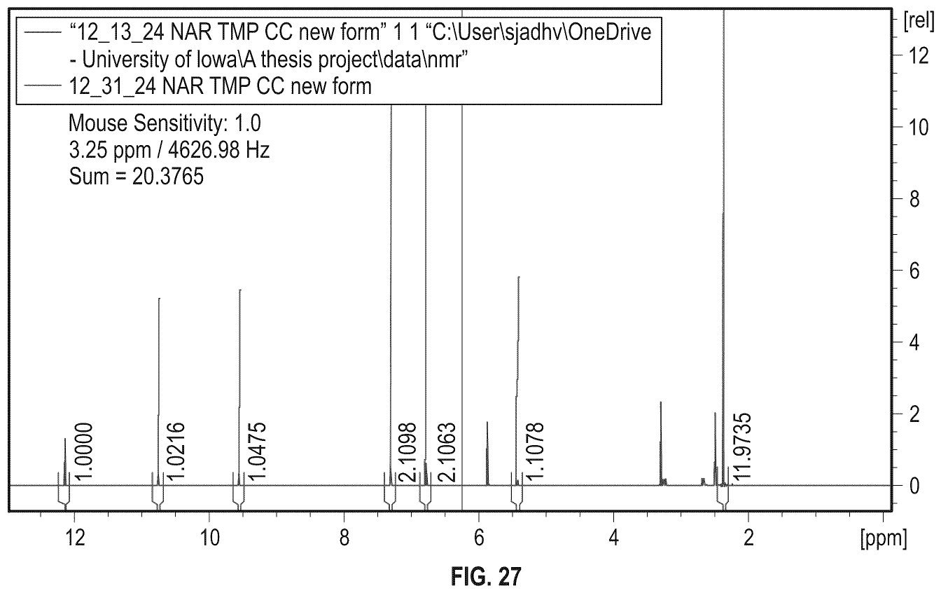

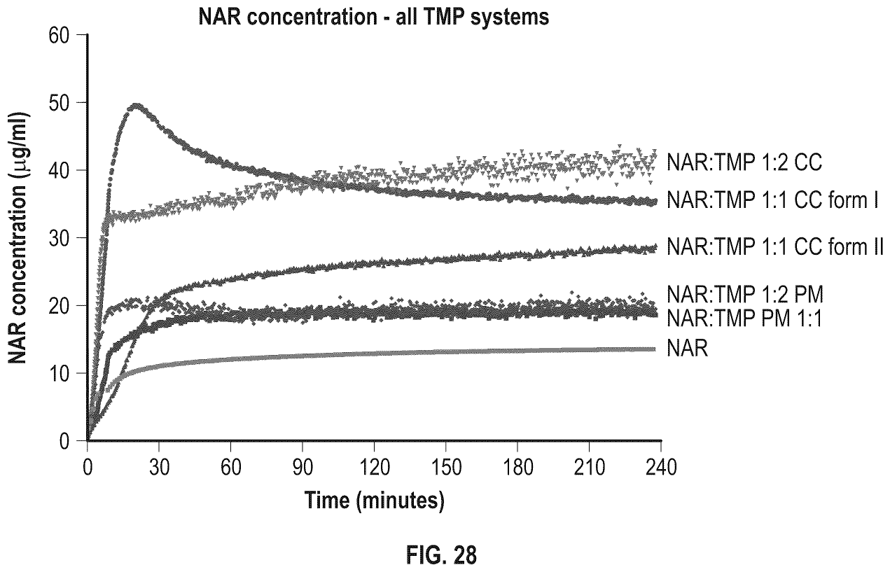

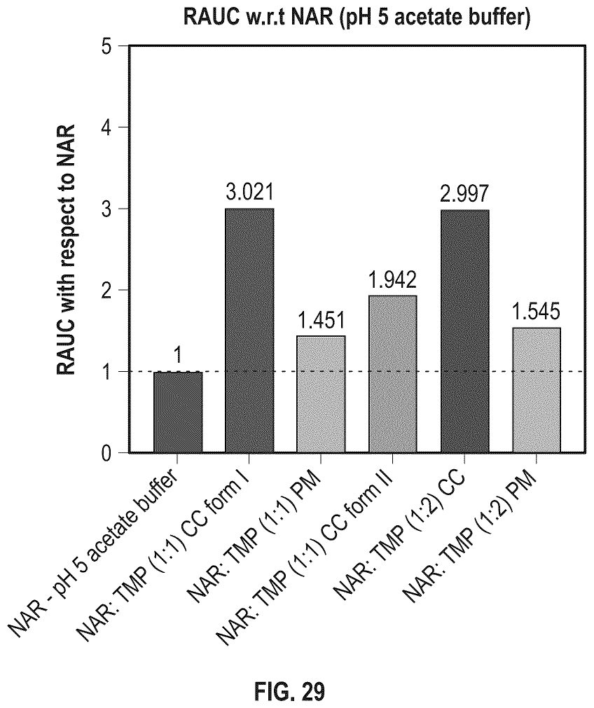

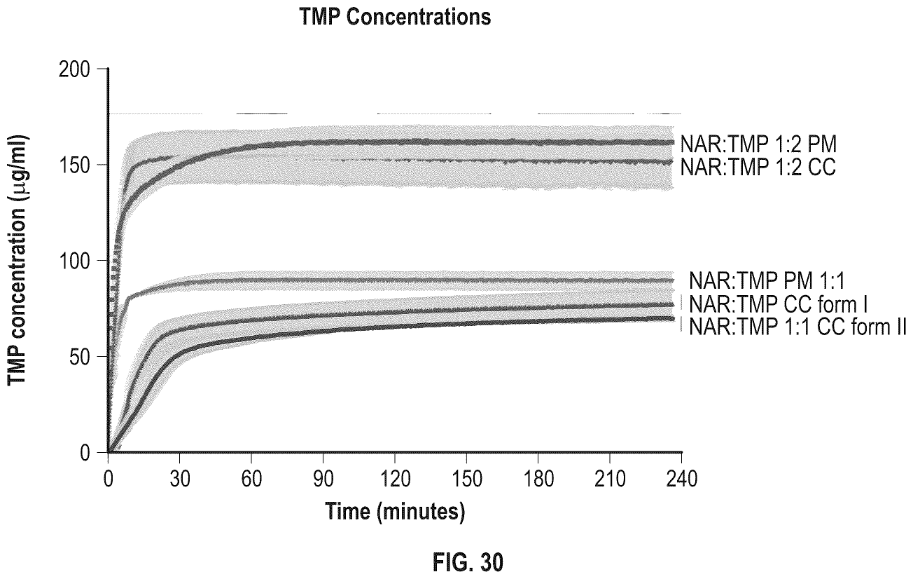

The solvent evaporation of equimolar ratio of NAR and betaine was carried out in 20-mL glass vial and the cap was loosely placed on the top of the vial for slow evaporation of the solvent. Two milliliters of methanol solvent were used to solubilize the drug:co-former system and the evaporation was performed at room temperature in vibration free zone. The obtained single crystal was analyzed using SCXRD and the new co-crystal methanol solvate of NAR:Betaine was confirmed (see FIG. 2)

### Example 4. Preparation of Hesperetin-Tetramethyl Pyrazine Co-Crystal

The slurry method was employed to generate the co-crystal of Hesperetin with TMP. The nature of the solvent did not influence the formation of co-crystal; however, the purity of the new phase was dictated by the type of solvent. Methanol, ethanol, ethylacetate and IPA was used to optimize the slurry method to get a pure co-crystal phase. The equimolar ratio of drug:coformer system in 2 ml of ethyl acetate with total weight of 400 mg was stirred at room temperature for 72 hours which resulted in the pure HES:TMP co-crystal. The diffraction patterns of powder co-crystal showed non-overlapping characteristic peaks at 8.7°, 9.8° and 10.3°. The DSC analysis demonstrated two endotherms, first one around 180° C. owing to melting of co-crystal and another broad endotherm at 220° C. which could be attributed to melting of the excess hesperetin crystallized soon after co-crystal melting.

### Example 5. Preparation of Quercetin-Succinimide Co-Crystal

The co-crystal was produced using slurry method with methanol. The equimolar ratio of quercetin and succinimide with total weight of 200 mg were added into 1 ml of methanol such that both drug and co-former are above their solubility. The sample was stirred on rotatory shaker for 72 hours at room temperature. The sample was filtered and solid was dried at 70 C in oven for 24 hours. The characteristic distinguishable peaks were observed in diffraction spectra at 6.9°, 8.9°, 9.1° and 10.16° confirming the formation of new co-crystal phase.

DSC analysis showed three endotherms for a co-crystal phase wherein the first one associated with dehydration temperature, second one around 240° C. attributed to co-crystal melting and third around 325° C. which could be melting of excess quercetin generated after co-crystal melting. The solution-state ¹H NMR was used to determine the stoichiometry of the co-crystal phase. Two peaks, one at 12.5 ppm corresponding to one proton for pure quercetin and another peak around 2.2 ppm associated to four protons of succinimide, were compared in co-crystal sample to determine the stoichiometry. The NMR spectrum suggested 4:1 stoichiometry for quercetin:succinimide co-crystal.

The co-crystals of the invention are useful as a combination therapy against multiple diseases including Alzheimer's disease, diabetes, and cancer owing to their neuroprotective, anti-oxidant and anti-inflammatory activities.

### Example 6. Preparation of Naringenin-aspartame co-crystal

The liquid assisted grinding and slurry methods produced a pure co-crystal phase. Co-crystal was obtained with all of the following solvents: ethyl acetate, isopropyl alcohol, methanol, ethanol and acetonitrile. In case of LAG experiments, nature of solvent did not influence the formation and purity of the co-crystal. Ethyl acetate, isopropyl alcohol, methanol solvent resulted in the same co-crystal phase. Methanol and ethanol-based slurries were also resulted in same co-crystal phase as LAG method. However, slurries prepared in isopropyl alcohol and ethyl acetate were not successful in generating co-crystal and instead resulted starting components. The formation of co-crystal and stoichiometry was confirmed by PXRD, DSC, FT-IR and solution-state ¹H NMR. The overlaid diffraction patterns of NAR-ASP physical mixture and co-crystal showed non-overlapping characteristic peaks at 5.6°, 7.8° and 16.9°, 26.2° and 26.8° for co-crystal phase and thus confirming generation of new co-crystal phase.

Thermal analysis of co-crystal demonstrated three endothermic peaks at temperatures different from melting points of starting materials suggesting formation of new phase. However, presence of multiple endothermic peaks for pure co-crystal phase is atypical and thus FT-IR analysis was carried out to further support generation of pure co-crystal phase. The FT-IR analysis showed emergence of new peaks or shifting of peaks for co-crystal phase compared to physical mixture at 1745 cm⁻¹, to 1615 cm⁻¹ from 1599 cm⁻¹, to 1498 cm⁻¹ from 1490 cm⁻¹, to 1471, 1460 cm⁻¹ from 1453, 1456 cm⁻¹, to 1373 cm⁻¹ from 1382 cm⁻¹. Multiple shifts in FT-IR peaks for LAG sample compared to physical mixture strongly complements formation of co-crystal phase. The stoichiometric ratio of NAR and ASP in co-crystal was determined to be 1:2by ratioing integration of peaks corresponding to NAR and ASP using ¹H NMR spectroscopy. A peak at 5.7 ppm corresponds to 2 hydrogen atoms of NAR whereas 8.4 and 4.13 ppm individually corresponds to one hydrogen atom of aspartame.

### Example 7. Preparation of Naringenin-Hexamine Co-Crystal

An equimolar ratio of NAR and hexamine using liquid assisted grinding (LAG) and slurry methods produced a pure co-crystal phase. Co-crystal was obtained from LAG method with methanol and using slurry methods from ethyl acetate, isopropyl alcohol. The same cocrystal form was generated from both LAG and slurry methods. Slurries prepared in methanol and ethanol were not successful in generating co-crystal and instead resulted starting components. The formation of co-crystal was confirmed by PXRD and DSC. The overlaid diffraction patterns of NAR-Hexamine physical mixture and co-crystal showed four non-overlapping characteristic peaks in the range of 13 to14°, and a couple of distinguishing peaks around 18-19° for co-crystal phase and thus confirming generation of new co-crystal phase. The thermal analysis of co-crystal demonstrated a single endothermic peak at 180.40° C. which was different from melting points (253° C. for NAR and 273.49° C. for hexamine) of starting materials suggesting formation of new phase. A small endothermic peak observed after melting of cocrystal was likely due to degradation of sample. Interestingly, the melting point of co-crystal is substantially lower than that of melting points of both starting materials highlighting potential of co-crystal in improving solution behavior of NAR via co-crystallization. The stoichiometric ratio of NAR:Hexamine in co-crystal was determined using ¹H NMR spectroscopy and we suggest equimolar ratio of NAR and hexamine in the co-crystal.

### Example 8

The following illustrate representative pharmaceutical dosage forms, containing a co-crystal of the invention (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A co-crystal comprising a flavonoid other than naringenin and a co-crystal former.

2. The co-crystal of claim 1, wherein the co-crystal former is selected from the group consisting of succinimide, tetramethyl pyrazine, aspartame, hexamine, and betaine methanol.

3. The co-crystal of claim 1, wherein the flavonoid is hesperetin or quercetin.

4. The co-crystal of claim 1, wherein the flavonoid and a co-crystal former are present in a ratio of about 1:1 or about 1:2.

5. A co-crystal comprising a flavonoid and a co-crystal former selected from the group consisting of succinimide, tetramethyl pyrazine, aspartame, hexamine, and betaine methanol.

6. The co-crystal of claim 5, wherein the flavonoid is hesperetin, naringenin, or quercetin.

7. The co-crystal of claim 5, wherein the flavonoid and a co-crystal former are present in a ratio of about 1:1 or about 1:2.

8. The co-crystal of claim 5, which comprises the flavonoid naringenin and the co-crystal former succinimide; the flavonoid naringenin and the co-crystal former tetramethyl pyrazine; the flavonoid naringenin and the co-crystal former betaine methanol, the flavonoid hesperetin and the co-crystal former tetramethyl pyrazine; the flavonoid quercetin and the co-crystal former succinimide; the flavonoid naringenin and the co-crystal former aspartame; or the flavonoid naringenin and the co-crystal former hexamine.

9. A pharmaceutical composition comprising a co-crystal of claim 1 and a pharmaceutically acceptable excipient.

10. A pharmaceutical composition comprising a co-crystal of claim 5 and a pharmaceutically acceptable excipient.

11. A method for treating cancer in an animal comprising administering a co-crystal of claim 1 to the animal, wherein the co-crystal has anticancer properties.

12. A method for producing an antioxidant effect in an animal in need of such treatment comprising administering a co-crystal of claim 1 to the animal, wherein the co-crystal has antioxidant properties.

13. A method for producing an anti-inflammatory effect in an animal in need of such treatment comprising administering a co-crystal of claim 1 to the animal, wherein the co-crystal has anti-inflammatory properties.

14. A method for treating cardiovascular disease in an animal comprising administering a co-crystal of claim 1 to the animal, wherein the co-crystal treats cardiovascular disease.

15. A method for improving cognitive function in an animal in need of such treatment comprising administering a co-crystal of claim 1 to the animal, wherein the co-crystal has cognitive enhancing properties; or a method for treating Alzheimer's disease in an animal comprising administering a co-crystal of claim 1 to the animal, wherein the co-crystal treats Alzheimer's disease.

16. A method for treating cancer in an animal comprising administering a co-crystal of claim 5 to the animal, wherein the co-crystal has anticancer properties.

17. A method for producing an antioxidant effect in an animal in need of such treatment comprising administering a co-crystal of claim 5 to the animal, wherein the co-crystal has antioxidant properties.

18. A method for producing an anti-inflammatory effect in an animal in need of such treatment comprising administering a co-crystal of claim 5 to the animal, wherein the co-crystal has anti-inflammatory properties.

19. A method for treating cardiovascular disease in an animal comprising administering a co-crystal of claim 6 to the animal, wherein the co-crystal treats cardiovascular disease.

20. A method for improving cognitive function in an animal in need of such treatment comprising administering a co-crystal of claim 5 to the animal, wherein the co-crystal has cognitive enhancing properties; or a method for treating Alzheimer's disease in an animal comprising administering a co-crystal of claim 5 to the animal, wherein the co-crystal treats Alzheimer's disease.
